(19) United States
(12) Patent Application Publication
(10) Pub. No.: US 2008/0181942 A1
(43) Pub. Date: Jul. 31, 2008

(54) Delta-9-THC compositions and methods for treating symptoms associated with multiple sclerosis

(75) Inventor: John Zajicek, Plymouth (GB)

Correspondence Address: Davidson, Davidson & Kappel, LLC, 485 7th Avenue, 14th Floor, New York, NY, 10018, US

(73) Assignee: University of Plymouth, Plymouth (GB)

(21) Appl. No.: 11/998,460

(22) Filed: Nov. 28, 2007

Related U.S. Application Data
(60) Provisional application No. 60/872,014, filed on Nov. 30, 2006.

(51) Int. Cl. A61K 9/48; A61K 31/352; A61P 25/00

(52) U.S. Cl. 424/456; 514/454

(57) ABSTRACT

Methods are provided for, inter alia, treating and/or preventing symptoms associated with multiple sclerosis and MS relapse.

This application claims the benefit of U.S. Provisional Patent Application No. 60/872,014, filed on Nov. 30, 2006, the disclosure of which is hereby incorporated by reference in its entirety.

### FIELD OF THE INVENTION

The present invention relates to methods of treating and/or preventing symptoms associated with multiple sclerosis (MS), and to methods of preventing MS relapse in a subject having MS.

### BACKGROUND OF THE INVENTION

Multiple sclerosis (MS) is believed to be an autoimmune disease that affects the central nervous system (CNS). The CNS consists of the brain, spinal cord, and the optic nerves. Surrounding and protecting the nerve fibers of the CNS is a fatty tissue called myelin that helps nerve fibers conduct electrical impulses. In MS, myelin is lost in multiple areas, leaving scar tissue called sclerosis. These damaged areas are also known as plaques or lesions. In some cases, the nerve fiber itself is damaged or broken. When myelin or the nerve fiber is destroyed or damaged, the ability of the nerves to conduct electrical impulses to and from the brain is disrupted, and this produces the various symptoms of MS.

Patients with MS can expect one of three main clinical courses of disease: relapsing-remitting, primary-progressive, and secondary-progressive. People with relapse-remitting MS experience clearly defined flare-ups (also called relapses, attacks, or exacerbations). These are episodes of acute worsening of neurologic function and are followed by partial or complete recovery periods (remissions) free of disease progression.

Patients with primary-progressive MS experience a slow but nearly continuous worsening of their disease from the onset, with no distinct relapses or remissions. However, there are variations in rates of progression over time, occasional plateaus, and temporary minor improvements.

Patients with secondary-progressive MS experience an initial period of relapsing-remitting disease, followed by a steadily worsening disease course with or without occasional flare-ups, minor recoveries (remissions), or plateaus.

Patients with MS also commonly experience one or more symptoms associated with MS including bladder or bowel dysfunction, problems with memory, attention, and problem-solving, dizziness and vertigo, depression, fatigue, balance problems, difficulty in walking, pain, sexual dysfunction, vision problems, hearing loss, headache, itching, seizures, spasticity, speech and swallowing difficulty, and/or tremor.

Muscle spasticity (stiffness resulting from increased pyramidal tone) and spasms occur in up to 90% of MS patients. This symptom often leads to considerable distress from pain, reduced mobility, and interference with activities of daily living. Other disabling features of the disease include ataxia and tremor in up to 80% of patients, and sensory symptoms, including pain, in up to 50% of MS patients. Lower urinary tract dysfunction is present in more than 90% of people with long-standing multiple sclerosis, with the most frequent symptoms being urinary frequency and urgency. Although many symptoms resolve in the remitting phase of multiple sclerosis, spasticity, weakness, ataxia, and bladder symptoms are often characteristic of progressive disease and tend to worsen over time.

Generally, there are two types of MS-related spasms: flexor and extensor. Flexor spasticity is an involuntary bending of the hips or legs (mostly involving the hamstring muscles on the back of the upper leg); the hips and knees bend up toward the chest. Extensor spasticity, on the other hand, is an involuntary straightening of the legs. Extensor spasticity involves the quadriceps and the adductors; the hips and knees remain straight with the legs very close together or crossed over at the ankles. Spasticity may also occur in the arms, but in MS this is less common.

Symptomatic MS therapy leaves much to be desired. For example, current treatments often provide inadequate symptom relief and are limited by toxicity. Existing treatments for spasticity in MS patients generally include baclofen, tizanidine, diazepam or clonazepam. Baclofen (Lioresal®) is a muscle relaxant that works in the spinal cord. Baclofen relaxes normal as well as spastic muscles and nausea is a common side effect.

Tizanidine (Zanaflex®) is a medication indicated for treatment of muscle spasticity. In addition to drowsiness, dry mouth is a common and usually temporary side effect. Hypotension (low blood pressure) is another potential side effect although less frequent. Moreover, tizanidine often causes greater sedation than other medications.

Spasticity has also been treated with diazepam (Valium®), often in small doses. Drowsiness and potential dependency with long-term use make diazepam undesirable for many patients.

Unfortunately, no single approved medication effectively treats MS-related spasms without unpleasant side-effects. A need therefore exists for a safe, effective method ameliorating symptoms experienced by patients with MS.

Dronabinol is a cannabinoid having the chemical designation (6aR-trans)-6a,7,8,10a-tetrahydro-6,6,9-trimethyl-3-pentyl-6H-dibenzo[b,d]pyran-1-ol and is also referred to as delta-9-tetrahydrocannabinol (delta-9-THC or Δ-9-THC). It is naturally occurring and has been extracted from Cannabis sativa L. (marijuana). It can also be chemically synthesized. Dronabinol is currently marketed under the trademark Marinol® for the treatment of anorexia associated with weight loss in patients with AIDS, and for the treatment of nausea and vomiting associated with cancer chemotherapy in patients who have failed to respond adequately to conventional antiemetic treatments. However, dronabinol is not currently approved for use in treating side effects associated with MS.

### SUMMARY OF THE INVENTION

It has now surprisingly been found that delta-9-THC can ameliorate symptoms of MS and can also reduce MS relapses. Accordingly, in one embodiment the present invention provides a method of treating and/or preventing side effects associated with MS. The method comprises administering to a subject suffering from MS a therapeutically effective amount of a cannabinoid, for example delta-9-tetrahydrocannabinol.

In another embodiment, the invention provides a method for reducing relapse-related hospital admissions in patients with MS.

In yet another embodiment, the invention provides a method for increasing mobility in a subject with MS.

According to methods of the present invention, a cannabinoid may be administered alone or in combination with one or more pharmaceutically effective carrier(s) or other pharmaceutically acceptable excipient(s) or additive(s).

In addition, a cannabinoid, for example dronabinol, can be administered concurrently or in succession with other medications, i.e. symptomatic therapies.

### BRIEF DESCRIPTION OF THE DRAWINGS

FIG. 1 shows changes in Ashworth scores from baseline to 13 weeks follow-up, adjusted for ambulatory status and center effects.

FIG. 2 shows effect of ambulation on Ashworth scores by treatment group.

FIG. 3 shows changes in Ashworth scores by visit and treatment group.

FIG. 4 shows median 10 meter walk times by visit and treatment group.—

### DETAILED DESCRIPTION

In one embodiment, the present invention provides methods for treating, limiting, ameliorating, reducing, delaying and/or improving symptoms associated with MS. The method according to this embodiment comprises administering to a subject in need thereof a therapeutically effective amount of a cannabinoid.

The term “cannabinoid” herein includes, inter alia, delta-8-tetrahydrocannabinol, delta-9-tetrahydrocannabinol, cannabidol, olivetol, cannabinol, cannabigerol, nabilone, and delta-9-tetrahydro cannabinotic acid. The non-psychotropic cannabinoid 3-dimethylnepty 11 carboxylic acid homologine 8, delta-8-tetrahydrocannabinol, (See e.g. J. Med. Chem. 35, 3135, 1992) as well as the prodrugs and pharmaceutically acceptable salts of cannabinoids are also suitable for the present invention and are included in the term “cannabinoid”. A suitable prodrug is THC-hemisuccinate.

When compositions are used in a “therapeutically-effective amount” according to the present invention, this means that the dose of the therapeutic agent (or agents) is such that a therapeutic level of agent is delivered to the bloodstream over the term that the composition is to be used. Such delivery is dependent on a number of variables including the time period for which the individual dosage unit is to be used, or the flux rate of the therapeutic agent into the systemic circulation of the subject. It will be understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of a disorder in accordance with the present invention. In terms of treatment protocols, it should be appreciated that the dosage to be administered will depend on several factors, including the particular agent that is administered, the route administered, the physical state of the particular agent, the condition of the particular subject, etc. For example, the term “therapeutically effective amount” herein means an amount of cannabinoid sufficient to treat, limit, ameliorate, prevent, reduce, delay and/or improve one or more symptoms associated with MS. Such an amount will vary widely from subject to subject and will depend on, inter alia, body weight, severity and type of side-effect, intra-subject variations in metabolism of the particular cannabinoid in question, and desired effect. Illustratively, the amount may be from about 0.01 to 35 mg/kg of body weight administered one to five times per day.

Toxicity and therapeutic efficacy of the therapeutic agents (and hence the dosing) of the inventive compositions can be determined by standard pharmaceutical procedures, for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In one embodiment of the present invention, compounds that exhibit large therapeutic indices are used. While compounds that exhibit toxic side effects may be used, care should be taken to design a delivery system that targets such compounds to the site of affected tissue in order to minimize potential damage to uninfected cells and, thereby, reduce side effects.

The dose administered to a subject, particularly a human subject, in the context of the present invention should be sufficient to affect a therapeutic response over a reasonable time frame. The dose will be determined by the strength of the particular compositions employed and the condition of the person, as well as the body weight of the person to be treated. The size of the dose also will be determined by the existence, nature, and extent of any adverse side-effects that might accompany the administration of a particular composition. A suitable dosage for internal administration is 0.01 to 100 mg/kg per day. A preferred dosage is 0.01 to 35 mg/kg per day. A more preferred dosage is 0.05 to 5 mg/kg per day. A suitable concentration of dronabinol in pharmaceutical compositions for oral administration is 0.05 to 15% (by weight). A preferred concentration is from 0.02 to 5%. A more preferred concentration is from 0.1 to 4%. More preferably, 0.03 to 0.06 mg/kg body weight per day is administered orally, and most preferably, a 2.5 mg oral dosage form is administered two times per day. The most preferred dosage for extracorporeal administration is in the range from about 0.1 mg/kg to 5 mg/kg of body weight per day. For the rectal, topical (including buccal and sublingual) or transdermal route of administration, the preferred dosage thereof (estimated as the base) is in the range 0.05 mg/kg to 20 mg/kg of body weight per day. Although dronabinol may be administered as needed, preferably, dronabinol is administered one to five times per day.

Where dronabinol is the active drug in a composition used according to methods of the instant invention, it is preferable that dronabinol will be present in such a composition in a total amount of about 0.5 mg to about 20 mg, preferably about 1 mg to about 15 mg, and more preferably about 2 mg to about 12 mg. Illustratively, such a composition will comprise about 2 mg, about 2.5 mg, about 5 mg or about 10 mg dronabinol.

A cannabinoid can be formulated in any suitable pharmaceutical composition for use in methods according to the present invention. Such compositions can include dosage forms designed for oral, buccal, sublingual, subcutaneous, transdermal, intramuscular or intravenous, rectal, topical or inhalation administration.

Pharmaceutical compositions suitable for use in methods of the present invention can include one or more conventional nontoxic pharmaceutically acceptable excipients such as fillers, binders, carriers, adjuvants, and/or vehicles as desired. Carrier materials that can be employed are any of those commonly used excipients in pharmaceutics and should be selected on the basis of compatibility with the cannabinoid being used and the release profile properties of the desired dosage form or composition. Non-limiting examples of suitable pharmaceutically acceptable excipients include binders, disintegration agents, filling agents, surfactants, pH correcting agents, stabilizers, lubricants, diluents, anti-adherents, glidants, carriers, etc.

Non-limiting examples of suitable binders include acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

Non-limiting examples of suitable disintegration agents include starches, pregelatinized corn starch, pregelatinized starch, celluloses, cross-linked carboxymethylcellulose, crospovidone, cross-linked polyvinylpyrrolidone, a calcium, a sodium alginate complex, clays, alginates, gums, or sodium starch glycolate, and any disintegration agents used in tablet preparations.

Non-limiting examples of suitable filling agents include lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Non-limiting examples of suitable surfactants include sodium lauryl sulfate, sorbitan monooleate, polyoxyethylene sorbitan monooleate, polysorbates, polaxomers, bile salts, glyceryl monostearate, Pluronic™ line (BASF), and the like.

Non-limiting examples of suitable pH correcting agents (buffers) include citric acid, succinic acid, fumaric acid, malic acid, tartaric acid, maleic acid, glutaric acid sodium bicarbonate and sodium carbonate and the like.

Non-limiting examples of suitable stabilizers include any antioxidation agents, buffers, or acids, and the like.

Non-limiting examples of suitable lubricants include magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

Non-limiting examples of suitable wetting agents include oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

Non-limiting examples of suitable diluents include lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

Non-limiting examples of suitable anti-adherents or glidants include talc, corn starch, DL-leucine, sodium lauryl sulfate, and magnesium, calcium, or sodium stearates, and the like.

Non-limiting examples of suitable pharmaceutically compatible carriers include acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's, The Science and Practice of Pharmacy (2000); Lieberman, H. A. and Lachman, L., Eds., Pharmaceutical Dosage Forms, Marcel Decker, New York, N.Y., 1980; and Liebeman et al., Pharmaceutical Dosage Forms (Volumes 1-3, 1990).

When a desired excipient serves as a diluent, it can be a solid, semi-solid or liquid material, which acts as a vehicle, carrier or medium for the active ingredient. Thus, compositions suitable for use in methods of the instant invention can be in the form of a tablet, pill, powder, lozenge, sachet, cachet, troche, suspension, emulsion, aerosol (as a solid or in a liquid medium), capsule (e.g. soft and hard gelatin or HPMC capsules), sterile packaged powder, dispensable powder, granule, or liquid.

Tablet dosage forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. In one embodiment of the present invention, the manufacturing processes may employ one or a combination of methods: (1) dry mixing, (2) direct compression, (3) milling, (4) dry or non-aqueous granulation, (5) melt granulation, or (6) fusion. Lachman et al., The Theory and Practice of Industrial Pharmacy (1986). Such tablets may also comprise film coatings, which disintegrate upon oral ingestion or upon contact with diluent.

Compressed tablets are solid dosage forms prepared by compacting a formulation containing an acid-labile pharmaceutical agent and/or buffering agent and/or excipient selected to aid the processing and improve the properties of the product. The term “compressed tablet” generally refers to a plain, uncoated tablet for oral ingestion, prepared by a single compression or by pre-compaction tapping followed by a final compression.

The tablets or pills suitable for use in methods of the present invention may be coated or otherwise compounded to provide a dosage form affording the advantage of improved handling or storage characteristics. For example, the tablet or pill can comprise an inner dosage and an outer dosage component, the latter being in the form of an envelope over the former.

Since a tablet may be used to form rapidly disintegrating tablets, chewable tablets, lozenges, troches or swallowable tablets; the intermediate formulations, as well as the process for preparing them, provide additional aspects of the present invention.

Effervescent tablets and powders may also be used in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid. When the salts are added to water, the acids and the base react to liberate carbon dioxide gas, thereby causing “effervescence.”

Liquid dosage forms may also be used in methods according to the present invention and include non-aqueous solutions; suitably flavored non-aqueous syrups; oil suspensions; and flavored emulsions with edible oils, such as cottonseed oil, sesame oil, coconut oil or peanut oil, as well as elixirs and similar pharmaceutical vehicles.

Many other types of release delivery systems are available and known to those of ordinary skill in the art. They include polymer-based systems, such as polylactic and polyglycolic acid, polyanhydrides and polycaprolactone; nonpolymer systems that are lipids, including sterols, such as cholesterol, cholesterol esters and fatty acids, or neutral fats, such as mono-, di- and triglycerides; hydrogel release systems; silastic systems; peptide-based systems; wax coatings; compressed tablets using conventional binders (See, for example, Lieberman et al., Pharmaceutical Dosage Forms, 2 Ed., Vol. 1, pp. 209-214 (1990), and excipients; partially fused implants; and the like. Specific examples include, but are not limited to: (a) erosional systems in which the polysaccharide is contained in a form within a matrix, found in U.S. Pat. No. 4,452,775; U.S. Pat. No. 4,667,014; and U.S. Pat. No. 4,748,034 and U.S. Pat. No. 5,239,660; and (b) diffusional systems in which an active component permeates at a controlled rate through a polymer, found in U.S. Pat. No. 3,832,253 and U.S. Pat. No. 3,854,480.

Formulations suitable for parenteral administration include aqueous and non-aqueous solutions, isotonic sterile injection solutions, which can contain anti-oxidants, buffers such as acetate and phosphate, toxicity adjusting agents, such as sodium chloride, pH adjusting agents, such as hydrochloric and phosphoric acid, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

In a preferred embodiment, dronabinol is administered according to methods of the invention as an aeorosolized formulation. Non-limiting examples of aerosolized formulations of dronabinol are disclosed in U.S. Pat. No. 6,509,005 to Peart et al., which is hereby incorporated by reference herein in its entirety.

In another preferred embodiment, dronabinol is administered as an oral capsule composition containing 2.5 mg, 5 mg or 10 mg dronabinol, sesame oil, gelatin, glycerin, methylparaben, propylparaben, and titanium dioxide.

Dronabinol may be administered in combination with one or more additional medications, for example medications used to treat symptoms of MS or to treat MS itself (disease-modifying agents). Non-limiting examples of medications that may be administered in combination with dronabinol include amantadine, baclofen, mineral oil, papaverine, meclizine (Antivert®), hydroxyzine (Atarax®), interferon-.quadrature.-1a (Avonex®), sulfamethoxazole (Bactrim®, Septra®), ciprofloxacin, (Cipro®), docusate (Colace®), glatiramer acetate (Copaxone®), pemoline (Cylert®), dantrolene (Dantrium®), desmopressin (DDAVP®), dexamethasone (Decadron®), prednisone (Deltasone®), tolterodine (Detrol®), phenyloin (Dilantin®), oxybutynin (Ditropan®), bisacodyl (Dulcolax®), venlafaxine (Effexor®), amitriptyline (Elavil®), docusate (Enemeez®), sodium phosphate, methenamine (Mandelamine®), Balcofen®, clonazepam (Klonopin®), isoniazid (Laniazid®), vardenafil (Levitra®), nitrofurantoin (Macrodantin®), psyllium hydrophilic mucilloid (Metamucil®), alprostadil, gabapentin (Neurontin®), mitoxantrone (Novantrone®), oxybutynin (Oxytrol®), nortiptyline (Pamelor®), paroxetine (Paxil®), propantheline bromide (Pro-Banthine®), alprostadil (Prostin® VR), modafinil (Provigil®), fluoxetine (Prozac®), phenazopyridine (Pyridium®), interferon-b-1a (Rebif®), glycerin (Sani-Supp®), methylprednisolone (Solu-Medrol®), carbamazepine (Tegretol®), imipramine (Tofranil®), diazepam (Valium®), sildenafil (Viagra®), bupropion (Wellbutrin®), tizanidine (Zanaflex®), and sertraline (Zoloft®).

The following examples illustrate embodiments of the present invention but should not be construed as limiting the scope of the instant invention in any way.

### DETAILED DESCRIPTION OF THE PREFERRED EMBODIMENTS

Features of the invention will become apparent in the course of the following descriptions of exemplary embodiments which are given for illustration of the invention and are not intended to be limiting thereof.

In the following examples, and throughout this specification, all parts and percentages are by weight, and all temperatures are in degrees Celsius, unless expressly stated to be otherwise. Where the solids content of a dispersion or solution is reported, it expresses the weight of solids based on the total weight of the dispersion or solution, respectively. Where a molecular weight is specified, it is the molecular weight range ascribed to the product by the commercial supplier, which is identified. Generally this is believed to be weight average molecular weight.

### Example 1

A study was performed to assess the use of cannabis extract and delta-9-THC in treating various symptoms associated with MS in a randomized, placebo-controlled study. Patients aged 18-64 years with clinically definite or laboratory-supported multiple sclerosis who had exhibited stable disease for the previous 6 months, with problematic spasticity (Ashworth score of greater than or equal to 2 in two or more lower limb muscle groups) were included in the trial. Patients with ischaemic heart disease, those with active sources of infection, and those taking medication such as beta interferon (that could impact spasticity) were excluded.

Patients were randomly assigned to receive one of two active treatments or placebo. Active treatment consisted of either synthetic delta-9-THC (Marinol, Solvay Pharmaceuticals, Atlanta, USA) or a cannabis extract, containing delta-9-THC and cannabidiol as the main cannabinoids (Cannador, Institute for Clinical Research, IKF, Berlin, Germany). Capsules were manufactured to contain 2.5 mg of delta-9-THC equivalent, 1.25 mg of cannabidiol, and less than 5% other cannabinoids per capsule. Medication was taken twice daily, after food. All other medication was taken as usual, except other oil-based capsules which were requested to be taken separately from trial medication to avoid possible interference with absorption.

The study started with a 5-week dose titration phase. During that period, patients increased their dose by one capsule (2.5 mg delta-9-THC equivalent) twice daily at weekly intervals. If side-effects developed, patients were advised not to increase the dose, and if side-effects were considered intolerable, the dose was reduced. Weeks 6-13 constituted a plateau phase, during which participants remained on a stable dose of medication (visits 5, 6, and 7). During week 14, patients reduced their medication by one capsule twice daily each day until they were off study medication. Patients remained off trial medication during week 15, and a final assessment was undertaken at the end of this week (visit 8).

The primary outcome measure of the study was change in spasticity related to multiple sclerosis, using the Ashworth score of spasticity (See, e.g., Ashworth, B. Preliminary trial of carisoprodol in multiple sclerosis. Practitioner 1964; 192: 540-42). Assessment of the Ashworth score was made at six visits: two pre-treatment (visits 1 and 2), three during treatment (visits 5, 6, and 7), and one after discontinuation of treatment (visit 8). The Ashworth score is an assessment of biological impairment and is dependent on the estimation of the physician. The score consists of a 5-point scale (0=normal, 1=slight catch when the limb is moved, 2=anything more than a catch but not restricting movement, 3=considerable increase in tone limiting passive flexion, 4=limb rigidity in flexion or extension). Ten muscle groups on each side of the body (elbow flexors, extensors, pronators and supinators; wrist and finger flexors; hip adductors, knee flexors and extensors, and foot plantar flexors) were assessed. Each patient was assessed supine on a couch, or as close to this position as was tolerated, after resting for 15 min. The limb being assessed was moved rapidly in the direction required by assessment. As spasticity can change with passive limb movement, the number of movements of each joint was kept to a minimum. The presence of more than seven beats of clonus on examining a joint was taken as implying at least grade 2 spasticity.

Table 1 provided immediately below provides baseline characteristics of the participants.

TABLE 1

| | Cannabis | | | | | |
|---|---|---|---|---|---|---|
| | Extract | | Delta-9-THC | | Placebo | |
| | No. | Mean | No. | Mean | No. | Mean |
| Sex | | | | | | |
| Male | 76 | — | 63 | — | 78 | — |
| Female | 135 | — | 143 | — | 135 | — |
| Age (years) | 211 | 50.5 | 206 | 502 | 213 | 50.9 |
| Height (cm) | 209 | 167.5 | 205 | 167.9 | 210 | 168.0 |
| Weight (kg) (n = 630) | 211 | 71.7 | 206 | 71.2 | 213 | 71.6 |
| Body-mass Index (kg/m2) | 209 | 25.6 | 205 | 25.2 | 210 | 25.4 |
| Mean Baseline Ashworth | | | | | | |
| Upper-body muscles | 211 | 5.0 | 206 | 5.9 | 212 | 5.4 |
| Lower-body muscles | 211 | 16.8 | 206 | 16.7 | 213 | 16.1 |
| All muscle groups | 211 | 21.8 | 206 | 22.5 | 213 | 21.4 |
| Form of MS | | | | | | |
| Relapsing/remitting | 6 | 3% | 14 | 7% | 13 | 6% |
| Primary progressive | 53 | 25% | 43 | 21% | 49 | 23% |
| Secondary progressive | 152 | 72% | 149 | 72% | 151 | 71% |
| Ambulatory status | | | | | | |
| Able to walk | 103 | 49% | 95 | 46% | 105 | 49% |
| Unable to walk | 108 | 51% | 111 | 54% | 108 | 51% |

Of the 630 patients included in the intention-to-treat analysis, follow-up data on the primary outcome was obtained for 611 (97%). Completion and return of data for the secondary outcome measures was also generally high, with data available for analysis from 84-91% of patients.

With respect to analysis of Ashworth scores, 81% (n=513) of patients had the same assessor throughout or had a different assessor at just one visit (cannabis extract 82% (n=173), delta-9-THC 82% (n=168), placebo 81% (n=172)). The primary outcome was defined as the change from baseline (mean of two baseline pre-treatment visits) to the end of the 13-week treatment period (visit 7). In accordance with the protocol, missing Ashworth scores at visit 7 were replaced by carrying forward the most recent Ashworth score available during the treatment phase. In total, 39 scores were carried forward; 28 from visit 6 and 11 from visit 5, distributed across treatments (12 cannabis extract, 17 delta-9-THC, 10 placebo). Primary outcome data were not available for 46 patients originally randomized (12 cannabis extract, 19 delta-9-THC, 15 placebo).

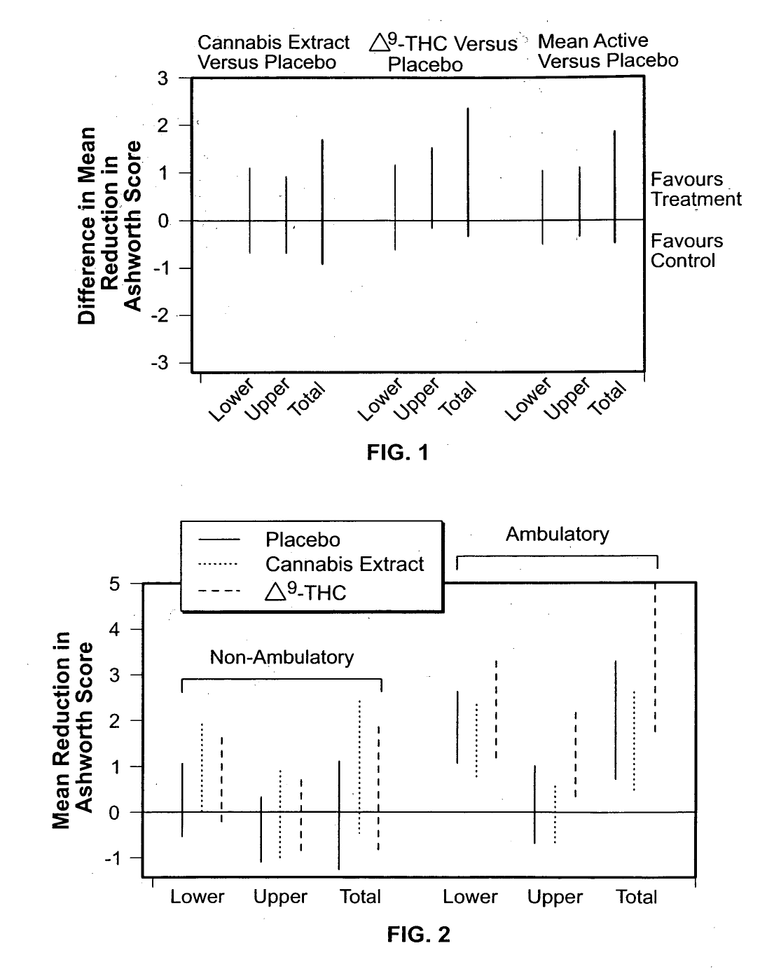

There was no statistically significant evidence of an effect of treatment on change in total Ashworth score from baseline to 13 weeks follow-up (p=0.29 with adjustment for ambulatory status and center, p=0.40 without adjustment). Mean (SD) changes in total Ashworth scores (baseline minus follow-up) were 1.24 (6.60), 1.86 (7.95), and 0.92 (6.56) for cannabis extract, delta-9-THC, and placebo, respectively. Corresponding figures for upper-body muscle groups were −0.05 (4.11), 0.48 (4.70), and −0.11 (4.04), and for lower-body muscle groups were 1.29 (4.37), 1.39 (5.21), and 1.04 (4.20). With both active treatments, an improvement over placebo was observed for the treatment effect when adjusted for center and for ambulatory status (See FIG. 1).

There was no statistically significant evidence of a treatment effect on changes in lower-body (adjusted for center and ambulatory status p=071, unadjusted p=074) or upper-body (p=020 and p=031) components of the Ashworth score, and no evidence of any interaction effect between center and treatment, between ambulatory status and treatment, or between baseline Ashworth score and treatment.

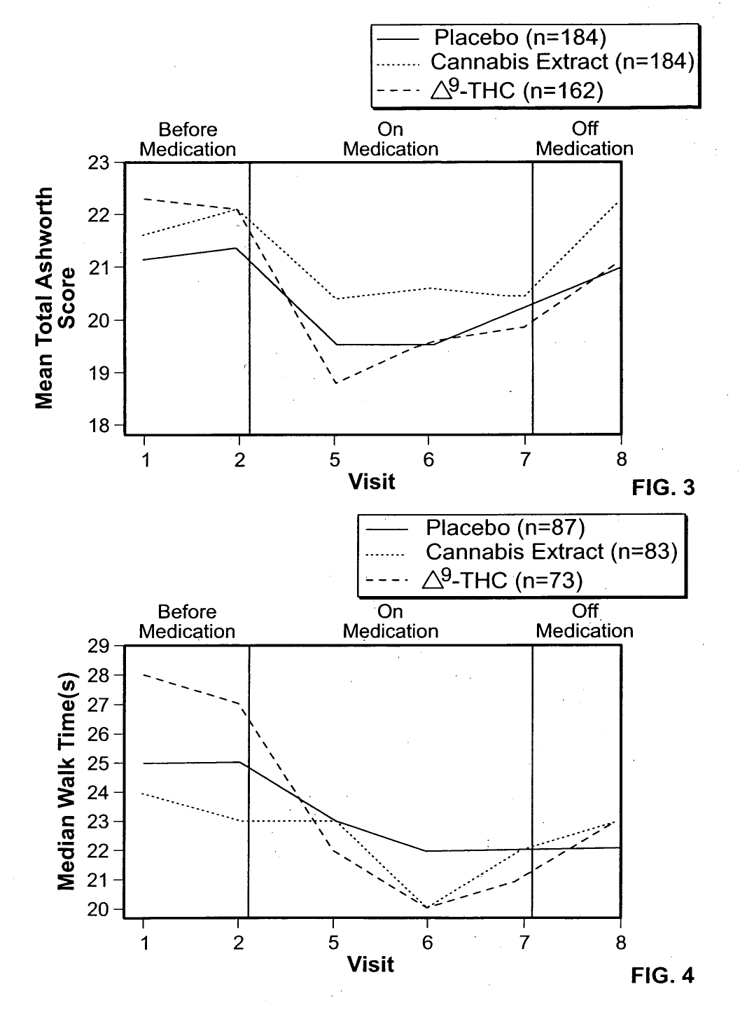

Both center (p<0.0001) and ambulatory status (p=0.002) had a significant effect on change in Ashworth score (FIG. 2). Estimated mean reduction in total Ashworth score for ambulatory patients relative to nonambulatory patients was 1.78 adjusted for treatment and center. There was also an improvement in the mean scores with treatment, occurring in all treatment groups, including placebo (FIG. 3).

### Example 2

Secondary outcomes were also measured in the above-described study. Such secondary outcomes included the Rivermead mobility index (See e.g. Collen, F. M. et al., The Rivermead mobility index: a further development of the Rivermead motor assessment. Int. Disanil. Stud. 1991; 13:50 54), a timed 10 meter walk, four self completion questionnaires—the United Kingdom neurological disability score (See e.g. Sharrack, B., Hughes R. A., The Guy's neurological disability scale (GNDS): a new disability measure for multiple sclerosis. Mult. Sder. 1999; 5: 223-33), and a series of nine category-rating scales. For the category-rating scale assessment, patients were asked to assess how their symptoms had been over the previous week compared with how they were just before the study started. Categories included irritability, depression, tiredness, muscle stiffness, tremor, pain, sleep, muscle spasms, and amount of energy. Data are discussed below.

With respect to secondary outcome measures, 322 patients provided at least one baseline walk time. Of these, seven (1 cannabis extract, 3 delta-9-THC, 3 placebo) dropped out of the trial. Walk times were obtained from 278 patients at visit seven. In total, 20 patients were unable to walk (8 cannabis extract, 5 delta-9-THC, 7 placebo) and very large walk times were substituted for these individuals.

Overall, a significant treatment effect on walk time from baseline to visit 7 (p=0.015) was observed. The median time taken to walk 10 meters was reduced from baseline to follow-up by 12% with delta-9-THC compared with a reduction with cannabis extract of 4% and placebo of 4%. FIG. 4 shows median walk time by visit and treatment group for patients who provided walk-time information at all six assessor visits.

Category rating scales were used to assess whether patients felt their symptoms had improved while on treatment relative to before start of treatment. Data are shown in Tables 2 and 3. Overall, patients felt that symptoms of pain, sleep quality, spasms, and spasticity had improved while on active treatment. No effect was noted with respect to irritability, depression, tiredness, tremor, or energy.

TABLE 2
Patient Reporting of Secondary Outcomes.

| | Improvement | | | Same | | | Deterioration | | |
|---|---|---|---|---|---|---|---|---|---|
| | Cannabis | Δ-9- | | Cannabis | Δ-9- | | Cannabis | Δ-9- | |
| | Extract | THC | Placebo | extract | THC | Placebo | extract | THC | Placebo |
| Irritability | 46 | 37 | 31 | 42 | 42 | 63 | 30 | 32 | 23 |
| | (39%) | (33%) | (26%) | (36%) | (38%) | (54%) | (25%) | (29%) | (20%) |
| Depression | 43 | 36 | 38 | 44 | 47 | 64 | 33 | 42 | 35 |
| | (36%) | (29%) | (28%) | (37%) | (38%) | (47%) | (28%) | (34%) | (26%) |
| Tiredness | 46 | 35 | 36 | 51 | 46 | 79 | 68 | 76 | 52 |
| | (28%) | (22%) | (22%) | (31%) | (29%) | (47%) | (41%) | (48%) | (31%) |
| Spasticity | 95 | 89 | 67 | 43 | 40 | 52 | 46 | 47 | 64 |
| | (52%) | (51%) | (37%) | (23%) | (23%) | (28%) | (25%) | (27%) | (35%) |
| Shake/tremor | 49 | 52 | 45 | 48 | 44 | 53 | 31 | 32 | 37 |
| | (38%) | (41%) | (33%) | (38%) | (34%) | (39%) | (24%) | (25%) | (27%) |
| Pain | 68 | 64 | 42 | 48 | 43 | 58 | 32 | 22 | 42 |
| | (46%) | (50%) | (30%) | (32%) | (33%) | (41%) | (22%) | (17%) | (30%) |
| Sleep | 82 | 71 | 59 | 62 | 57 | 79 | 20 | 24 | 25 |
| | (50%) | (47%) | (36%) | (38%) | (38%) | (48%) | (12%) | (16%) | (15%) |
| Spasms | 96 | 81 | 67 | 50 | 49 | 68 | 34 | 27 | 38 |
| | (53%) | (49%) | (39%) | (28%) | (29%) | (39%) | (19%) | (22%) | (22%) |
| Energy | 61 | 61 | 45 | 73 | 63 | 78 | 49 | 49 | 61 |
| | (33%) | (35%) | (24%) | (40%) | (36%) | (42%) | (27%) | (28%) | (33%) |

### Example 3

At visit 8 in study described in Examples 1 and 2, patients were asked specific questions about whether treatment had improved pain, tremor, spasticity, or bladder symptoms. Table 3 shows patient responses to those questions. Overall, more patients perceived an improvement in spasticity and pain when taking the active treatments than when taking placebo. Difference in perception of improvement in tremor was not statistically significant and no treatment effect on bladder symptoms was identified. Although there was no stratification for these specific symptoms between groups, the groups were broadly balanced for these symptoms apart from bladder symptoms, where there were fewer patients with urinary symptoms in the group taking, delta-9-THC.

There was a significant association between the actual treatment and the treating doctors' assessment of whether the patient was on active treatment (p<0.001). According to the treating doctors' assessment, 71% (n=140) of the cannabis extract group, 66% (n=119) of the delta-9-THC group, and 43% (n=85) of the placebo group were on active treatment. Similarly there was an association between the actual treatment and the patients' view of what they had taken (p<0.001). According to patients' reports, 77% (n=151), 77% (n=139), and 50% (n=98) of the cannabis extract, delta-9-THC, and placebo groups, respectively, thought that they had been on active treatment.

There was no association between the assessors' opinion of treatment and the actual treatment (p=0.72). The proportions viewed by the assessor as being on active medication in the three groups were 44% (n=90) cannabis extract, 39% (n=73) delta-9-THC, and 42% (n=86) placebo.

TABLE 3
Patient assessment of treatment benefit after week 8.

| Symptom | Cannabis Extract | Δ-9-THC | Placebo |
|---|---|---|---|
| Improvement | (n = 197) | (n = 181) | (n = 198) |
| Bladder | | | |
| Yes | 68 (44%) | 67 (40%) | 51 (33%) |
| No | 87 (56%) | 97 (59%) | 102 (67%) |
| Pain | | | |
| Yes | 83 (57%) | 64 (50%) | 51 (37%) |
| No | 63 (43%) | 64 (50%) | 86 (63%) |
| Tremor | | | |
| Yes | 58 (48%) | 44 (40%) | 43 (33%) |
| No | 64 (52%) | 67 (60%) | 89 (67%) |
| Spasticity | | | |
| Yes | 121 (61%) | 108 (60%) | 91 (46%) |
| No | 76 (39%) | 73 (40%) | 107 (54%) |

Unexpectedly, as can be seen in Table 4, incidences of MS relapse were greatly reduced in both the cannabis extract, and delta-9-THC groups by comparison with placebo.

TABLE 4
Adverse events reported by patients.

| Adverse Event | Cannabis Extract | Δ-9-THC | Placebo |
|---|---|---|---|
| MS relapse or possible relapse | 1 | 1 | 7 |
| Urinary tract infection | 1 | 3 | 4 |
| Pneumonia | 1 | 2 | 1 |
| Blocked/insertion of suprapubic | 1 | 0 | 3 |
| catheter | | | |
| Constipation | 1 | 0 | 3 |
| Grand mal seizure | 1 | 0 | 1 |
| Other | 6 | 11 | 2 |

What is claimed is:

1. A method of treating and/or preventing symptoms associated MS in a subject in need thereof, the method comprising administering to the subject a therapeutically effective amount of a pharmaceutical composition comprising delta-9-tetrahydrocannabinol.

2. The method of claim 1 wherein said administering step comprises oral administration.

3. The method of claim 1 wherein said delta-9-tetrahydrocannabinol is administered in the form of one or more capsules containing delta-9-tetrahydrocannabinol in sesame oil.

4. The method of claim 1 wherein the composition comprises a dosage form selected from the group consisting of an intranasal solution or suspension, an inhalant solution or suspension, a parenteral solution or suspension, a transdermal patch, a transdermal gel, and a transdermal cream.

5. The method of claim 1 wherein the composition is administered in a dosage form selected from the group consisting of a tablet, a capsule, an inhalant, an injectable, a transdermal, a sublingual, and a suppository.

6. The method of claim 5 wherein the composition is in the form of an inhalant.

7. The method of claim 6 wherein the inhalant is administered orally.

8. The method of claim 5 wherein the capsule is a soft gelatin or HPMC capsule.

9. The method of claim 1 wherein the composition is administered in combination with one or more MS therapies.

10. The method of claim 1 wherein the delta-9-tetrahydrocannabinol is dronabinol.

11. The method of claim 1 wherein the composition is administered to the patient by a route selected from the group consisting of oral, intranasal, inhalation, injection, transdermal, and sublingual.

12. The method of claim 11 wherein the inhalation comprises oral inhalation.

13. The method of claim 1 wherein the composition is administered in an amount sufficient to provide about 2.5 mg delta-9-tetrahydrocannabinol to about 20 mg delta-9-tetrahydrocannabinol per day.

14. The method of claim 1 wherein the composition is administered in an amount sufficient to provide about 2.5 mg delta-9-tetrahydrocannabinol per day.

15. The method of claim 1 wherein the composition is administered about 1 to about 4 times per day.

16. The method of claim 1 wherein the composition is administered in a single dose.

17. A method of preventing an MS relapse in a subject in need thereof, the method comprising administering to the subject a therapeutically-effective amount of a pharmaceutical composition comprising delta-9-tetrahydrocannabinol.
